(19) United States
(12) Patent Application Publication
(10) Pub. No.: US 2014/0276122 A1
(43) Pub. Date: Sep. 18, 2014

(54) METHOD AND SYSTEM FOR NEUROCARDIAC DIFFERENTIAL ANALYSIS OF ISCHEMIA AND MYOCARDIAL INFARCTION

(71) Applicant: PACESETTER, INC., Sylmar, CA (US)

(72) Inventors: Gary R. Dulak, Moorpark, CA (US); Laurence S. Sloman, West Hollywood, CA (US); Stuart Rosenberg, Castaic, CA (US); Lanitia Ness, Los Angeles, CA (US); Yelena Nabutovsky, Sunnyvale, CA (US); Kwangdeok Lee, Valencia, CA (US); Rupinder Bharmi, Canyon Country, CA (US); Gene A. Bornzin, Simi Valley, CA (US)

(73) Assignee: PACESETTER, INC., Sylmar, CA (US)

(21) Appl. No.: 13/829,711

(22) Filed: Mar. 14, 2013

(51) Int. Cl. A61B 5/04; A61N 1/365; A61B 5/0402

(52) U.S. Cl. CPC A61B 5/04012; A61B 5/0402; A61N 1/36585
USPC 600/483; 607/62; 607/46

(57) ABSTRACT

A method and system for differential analysis of cardiac events are provided that include monitoring cardiac signals from a heart to detect deviations indicative of at least one of ischemia and myocardial infarction (MI). The method and system also monitor physiologic surrogate signals associated with pain to detect chest pain. Additionally, the method and system include characterizing a cardiac event exhibited by the heart based on whether the cardiac event occurs in a presence of at least one of the ischemia, IM, and chest pain.

### BACKGROUND OF THE INVENTION

Embodiments of the present invention generally relate to neurocardiac systems, and more particularly to neurocardiac systems that perform differential analysis of neurologic and cardiac signals.

Cardiac ischemia is a condition whereby the heart tissue does not receive adequate amounts of oxygen that is usually caused by a blockage of an artery leading to the heart tissue. Ischemia arises during angina, coronary angioplasty, and any other condition that compromises blood flow to a myocardial region. When blockage of an artery is sufficiently severe, the cardiac ischemia becomes a myocardial infarction (MI), which is also referred to as a heart attack.

Many patients at risk of various heart conditions, such as cardiac ischemia, have medical devices implanted therein. An implantable medical device is implanted in a patient to monitor, among other things, electrical activity of a heart and to deliver appropriate electrical therapy, neurostimulation, and/or drug therapy, as required. Implantable medical devices (“IMDs”) include for example, pacemakers, cardioverters, defibrillators, implantable cardioverter defibrillators (“ICDs”), and the like. The electrical therapy produced by an IMD may include, for example, pacing pulses, cardioverting pulses, and/or defibrillator pulses to reverse arrhythmias (e.g., tachycardias and bradycardias) or to stimulate the contraction of cardiac tissue (e.g., cardiac pacing) to return the heart to its normal sinus rhythm.

Additionally, IMDs may be used to deliver neurocardiac (NC) therapy in the form of neurostimulation (NS) in the thoracic region by spinal cord stimulation, transcutaneous electrical nerve stimulation, and/or subcutaneous electrical nerve stimulation. These NC treatments have been shown to have beneficial cardiovascular effects and also can treat angina. Presently, NC therapies for cardiac applications, including refractory angina, heart failure, arrhythmia, etc., are delivered either on a pre-determined schedule or on demand by a patient programmer. In addition, physicians may be concerned that treating angina pain with NS might conceal or mask pain associated with an MI, leading to a misdiagnosis or a delayed diagnosis. As such, physicians may be reluctant to use neurocardiac interventions without additional mitigation.

A need remains for an effective, automatic system for administering NC therapy to treat angina pain while reducing the risk of concealing pain associated with MI.

### SUMMARY

In accordance with one embodiment, a method is provided for differential analysis of cardiac events. The method includes monitoring cardiac signals from a heart to detect deviations indicative of at least one of ischemia and myocardial infarction (MI). The method also monitors physiologic surrogate signals associated with pain to detect chest pain. Additionally, the method includes characterizing a cardiac event exhibited by the heart based on whether the cardiac event occurs in a presence of at least one of the ischemia, MI, or chest pain.

Optionally, the monitoring of cardiac signals may include sensing at least one of cardiac electrical signals, cardiac impedance, and hemodynamic surrogate signals. The monitoring of cardiac signals may include monitoring ST levels in the cardiac signals for ST deviations. The monitoring of physiologic surrogate signals may sense at least one of blood pressure, heart rate, temperature, and respiration. The method may further include detecting the at least one of ischemia and MI based at least in part on a least one of: i) severity of ST deviation; ii) duration of ST deviation; and iii) changes in at least one of heart rate, heart rate variability, or heart rate morphology. Detecting the chest pain may be based at least in part on at least one of: i) heart rate; ii) temperature; iii) respiration rate; iv) chest constriction; or v) perspiration. The method may further provide a manual activator configured to permit a patient to designate the presence of the chest pain.

Optionally, the method for differential analysis of cardiac events further includes, delivering a neurocardiac (NC) therapy. Upon detection of ischemia, the method may deliver a neurocardiac (NC) therapy to treat the ischemia. Upon detection of the presence of chest pain, the method may issue an alert and generate a report based on whether at least one of ischemia and MI are present concurrent with the chest pain. When chest pain is detected and no ischemia is detected, the method may deliver a pain therapy. The method may further include delivering a NC therapy when chest pain is detected and ischemia is detected but no MI is detected. When chest pain, ischemia, and MI are detected, the method may deliver a NC therapy configured to provide an anti-ischemic effect without fully masking MI-related pain. The method may further include delivering NC therapy, wherein parameters of the NC therapy are adjusted based on a severity of the ischemia or MI and based on detection of the physiologic surrogate signals indicative of pain.

In accordance with one embodiment, a neurocardiac device (NCD) system is provided for differential analysis of cardiac events. The NCD system includes a cardiac signal monitoring (CSM) module configured to monitor cardiac signals from a heart to detect deviations indicative of at least one of ischemia and MI. The NCD system also includes a pain signal monitoring (PSM) module configured to monitor physiologic surrogate signals associated with pain to detect chest pain. Additionally, the NCD system includes a cardiac event characterization (CEC) module configured to characterize a cardiac event exhibited by the heart based on whether the cardiac event occurs in the presence of at least one of the ischemia, MI and chest pain.

Optionally, the NCD system has a neurocardiac therapy (NCT) module configured to deliver a NC therapy based on the cardiac event characterized by the CEC module.

### BRIEF DESCRIPTION OF THE DRAWINGS

FIG. 1 illustrates a neurological stimulation (NS) system according to an embodiment.

FIGS. 2A-2C illustrate stimulation portions for inclusion at the distal end of a lead in the NS system of FIG. 1.

FIG. 3 illustrates a diagram of an exemplary system that may be implemented in accordance with an embodiment.

FIG. 4 illustrates a flowchart of a detection process for differential analysis of cardiac events according to an example embodiment.

FIG. 5 illustrates a flowchart of an embodiment of a treatment process for differential analysis of cardiac events.

FIG. 6 illustrates a treatment process for differential analysis of cardiac events as shown in a matrix according to an example embodiment.

FIG. 7 illustrates a block diagram of an implantable medical device in accordance with an embodiment.

FIG. 8 illustrates a block diagram of an external programmer in accordance with an embodiment.

The foregoing summary, as well as the following detailed description of certain embodiments of the present invention, will be better understood when read in conjunction with the appended drawings. To the extent that the figures illustrate diagrams of the functional blocks of various embodiments, the functional blocks are not necessarily indicative of the division between hardware and circuitry. Thus, for example, one or more of the functional blocks (e.g., processors or memories) may be implemented in a single piece of hardware (e.g., a general purpose signal processor or a block or random access memory, hard disk, or the like). Similarly, the programs may be stand alone programs, may be incorporated as subroutines in an operating system, may be functions in an installed imaging software package, and the like. It should be understood that the various embodiments are not limited to the arrangements and instrumentality shown in the drawings.

### DETAILED DESCRIPTION

FIG. 1 depicts a neurological stimulation (NS) system 100 that generates electrical pulses for application to nervous tissue of a patient according to an embodiment. For example, the system 100 may be adapted to stimulate spinal cord tissue, peripheral nerve tissue, deep brain tissue, cortical tissue, and/or any other nervous tissue within a patient's body.

System 100 includes an implantable NS device 150 that is adapted to generate electrical neurostimulation (NS) pulses for application to the nerve system of a patient. The NS pulses may be part of a neurocardiac (NC) therapy designed to treat cardiac events, such as ischemia, and/or resolve or at least reduce chest pain. For this reason, the NS device 150 may also be referred to herein as a neurocardiac (NC) device. The NS device 150 may be implanted in a patient subcutaneously, transcutaneously, or may be fully implantable. Implantable NS device 150 typically comprises a metallic housing that encloses controller 151, pulse generating circuitry 152, battery 153, recharging circuit 154, far-field and/or near field communication circuitry 155, battery charging circuitry 156, switching circuitry 157, etc. of the device. Controller 151 may be configured to control at least some of the circuitry (e.g., 152, 154, 155, 156, 157) used to generate stimulation pulses during NC therapy. The NS device 150 may include a microcontroller 162 and/or other suitable processor for controlling at least some of the various other components of the device, such as communications between the NS device 150 and sensing leads and/or external devices. Software code is typically stored in memory 164 of the NS device 150 for execution by the controller 151, microcontroller 162, and/or other processor to control the various components of the device 150.

The NS device 150 may comprise a separate and/or an attached extension component 170. If extension component 170 is a separate component, extension component 170 may connect with the “header” portion (not shown) of NS device 150. If extension component 170 is integrated with NS device 150, internal electrical connections may be made through respective conductive components. Within NS device 150, electrical pulses are generated by pulse generating circuitry 152 and are provided to switching circuitry 157. The switching circuit 157 connects to outputs of NS device 150. Electrical connectors (e.g., “Bal-Seal” connectors) within connector portion 171 of extension component 170 and/or within the NS device 150 header may be employed to conduct the NS pulses. The terminals of one or more stimulation leads 110 are inserted within connector portion 171 and/or within the NS device 150 header for electrical connection with respective connectors. Thereby, the pulses originating from NS device 150 are provided to stimulation lead 110. The pulses are then conducted through the conductors of lead 110 and applied to tissue of a patient via electrodes 111 as neurostimulation. Any suitable known or later developed design may be employed for connector portion 171.

For implementation of the components within NS device 150, a processor and associated charge control circuitry for an implantable pulse generator are described in U.S. Patent Publication No. 20060259098, entitled “Systems and methods for use in pulse generation,” which is incorporated herein by reference. Circuitry for recharging a rechargeable battery of an implantable pulse generator using inductive coupling and external charging circuits are described in U.S. patent Ser. No. 11/109,114, entitled “Implantable device and system for wireless communication,” which is incorporated herein by reference. Various sets of parameters may define the pulse characteristics and pulse timing for the NS pulses applied to various electrodes as is known in the art. Although constant current pulse generating circuitry is contemplated for some embodiments, any other suitable type of pulse generating circuitry may be employed, such as constant voltage pulse generating circuitry.

Stimulation lead(s) 110 may comprise a lead body of insulation material about a plurality of conductors within the material that extend from a proximal end of lead 110 to its distal end. The conductors electrically couple a plurality of electrodes 111 to a plurality of terminals (not shown) of lead 110. The terminals are adapted to receive electrical pulses and the electrodes 111 are adapted to apply NS pulses to tissue of the patient. Stimulation lead 110 may include any suitable number of electrodes 111, sensors, terminals, and internal conductors.

FIGS. 2A-2C illustrate stimulation portions 200, 225, and 250 for inclusion at the distal end of lead 110. Stimulation portion 200, shown in FIG. 2A, depicts a conventional stimulation portion of a “percutaneous” lead with multiple ring electrodes. Stimulation portion 225 of FIG. 2B depicts a stimulation portion including several “segmented electrodes” 121. The term “segmented electrode” 121 is distinguishable from the term “ring electrode.” As used herein, the term “segmented electrode” refers to an electrode 121 or a group of electrodes 121 that are positioned at the same longitudinal location along the longitudinal axis of a lead and that are angularly positioned about the longitudinal axis so they do not overlap and are electrically isolated from one another. Example fabrication processes are disclosed in U.S. patent application Ser. No. 12/895,096, entitled, “Method of fabricating stimulation lead for applying electrical stimulation to tissue of a patient,” which is incorporated herein by reference. Stimulation portion 250 of FIG. 2C includes multiple planar electrodes 121 on a paddle structure.

Although not required for all embodiments, the lead bodies of lead(s) 110 and extension component 170 may be fabricated to flex and elongate in response to patient movements upon implantation within the patient. By fabricating lead bodies according to some embodiments, a lead body or a portion thereof is capable of elastic elongation under relatively low stretching forces. Also, after removal of the stretching force, the lead body is capable of resuming its original length and profile. For example, the lead body may stretch 10%, 20%, 25%, 35%, or even up to or above 50% beyond its original length at forces of about 0.5, 1.0, and/or 2.0 pounds of stretching force.

Referring again to FIG. 1, a sensing lead 140 is connected to the NS device 150. The sensing lead 140 collects cardiac and/or physiologic surrogate signals from a patient and conveys the signals, through conductors in the lead 140, to one or more inputs 161 of the NS device 150. The sensing lead 140 includes sensors 142 and 144 that sense cardiac and/or physiologic activity and generate cardiac and/or physiologic surrogate signals associated therewith. For example, the sensors 142, 144 may sense cardiac signals such as intracardiac electrogram (IEGM) signals, electrocardiogram (ECG) signals, heart rate signals, blood pressure signals, blood oxygen content signals, and the like. The ECG signals are composed of various waves and segments that represent the heart depolarizing and repolarizing, and are useful for diagnosing certain heart conditions, such as ischemia and locating damaged areas within the heart. The sensors 142, 144 may additionally or alternatively sense physiologic surrogate signals such as blood pressure, heart rate, temperature, perspiration, respiration rate, etc.

The NS device 150 includes one or more inputs 161 that are configured to receive cardiac and/or physiologic surrogate signals. The inputs 161 may direct the received signals to the controller 151, microcontroller 162, other processor, or the like, for processing and further action. Optionally, the inputs 161 may also receive cardiac and/or physiologic surrogate signals from one or more separate implantable medical devices (IMD) and/or external medical devices (EMD).

In an alternative embodiment, one lead may be configured as both a stimulation lead and a sensing lead. For example, a single lead may include both electrodes and sensors at distal or intermediate portions thereof. Conductors within the lead may carry electrical pulses from the NS device 150 to the electrodes, where the pulses are converted to neurostimulation applied to the tissue of the patient. Separate conductors within the lead may relay received cardiac and/or physiological surrogate signals from the sensors to the inputs 161 of the NS device 150 for processing. Alternatively, in an example embodiment, one or more electrodes along the lead may be configured to both convey neurostimulation to the tissue of a patient as NC therapy and also monitor cardiac and/or physiologic surrogate signals from the patient.

The NS device 150 may be configured for differential analysis of cardiac events. For example, the NS device 150 may analyze received signals, such as cardiac and/or physiologic surrogate signals, to identify the occurrence of an MI, ischemia, and/or chest pain, which collectively or individually are referred to herein as cardiac events. For example, a declared cardiac event may include without limitation one or more of an MI, ischemia, or chest pain. Once a cardiac event is declared, depending on the characteristics of the cardiac event the NS device 150 may be configured to implement a NC therapy, notify emergency services to respond to the patient, and/or alert the patient, among other actions. The controller 151 and/or microcontroller 162 may be programmed to declare a particular cardiac event based on the received cardiac and/or physiologic surrogate signals. In addition, the controller 151 and/or microcontroller 162 may be programmed to direct the NS device 150 to begin NC operation in a select one of programmed NS parameters based on the particular cardiac event declared. Alternatively, the selected NS parameters may be preprogrammed and not dependent on the particular cardiac event.

The NS device 150 includes memory 164 that is configured to save multiple NS parameters. The memory 164 maintains a one to one relation between the cardiac and/or physiologic surrogate signals that are derived and the NS parameters operative at the time when the signals were collected. Upon declaration of a particular cardiac event, the controller 151 and/or microcontroller/processor 162 may select certain NS parameters stored in the memory 164 to use in delivering NC therapy to the patient. Thus, the memory 164 may be configured to store pre-programmed executable software code, collect and store cardiac and/or physiologic surrogate signals, and keep a record of the NS parameters used during NC therapy. The memory 164 may also store/record other activities in response to declared cardiac events, including generated clinician reports, patient alerts, and EMS notifications.

In an embodiment, the NS device 150 includes a cardiac signal monitoring (CSM) module 158 configured to monitor cardiac signals from a heart to detect deviations indicative of at least one of ischemia and MI. The CSM module 158 may continuously or repetitiously receive cardiac signals collected by the electrodes 111 and/or sensors 142, 144. Based on the data received, the CSM module 158 may detect changes in at least one of heart rate, heart rate variability, heart rate morphology, ST segment, and the like. For example, the CSM module 158 may detect onset and/or termination of ST segment shift/deviation in an ECG, including severity and duration of the ST deviation. The ST segment, displayed in ECGs, represents the portion of the cardiac signal between ventricular depolarization and ventricular repolarization. Heart conditions may be detected by identifying variations in the ST segment from the baseline cardiac signal that occurs during ST episodes (i.e. cardiac events). For example, ST segment shifts may be measured and compared to an ST threshold to identify cardiac events and/or a potential abnormal physiology (e.g., an MI, ischemia, a heart block, an arrhythmia, fibrillation, congestive heart failure, and the like). The CSM module 158 may be a processing unit within the controller 151 or microcontroller 162, or may be a processor within the NS device 150. The CSM module 158 may include one or more sensors, electrodes, monitors, associated software, associated circuitry, and the like.

The NS device may also include a pain signal monitoring (PSM) module 163 configured to monitor physiologic surrogate signals associated with pain to detect chest pain. The PSM module 163 may monitor physiologic surrogate signals, such as heart rate, blood pressure, temperature, chest constriction, respiration rate, perspiration, and the like. Based on the sensed physiologic surrogate signals, the PSM module 163 may be able to detect the presence of chest pain. The PSM module 163 may be a processing unit within the controller 151 or microcontroller 162, or may be a processor within the NS device 150. The PSM module 163 may include one or more sensors, electrodes, monitors, associated software, associated circuitry, and the like.

Additionally, the NS device 150 may include a cardiac event characterization (CEC) module 167 configured to characterize a cardiac event exhibited by the heart based on whether the cardiac event occurs in a presence of at least one of ischemia, MI, and chest pain. The CEC module 167 may make a differential analysis of a cardiac event based on information received from the CSM module 158 and/or the PSM module 163. More specifically, the cardiac and physiologic surrogate information that is monitored by the CSM and PSM modules 158, 163 may be conveyed to the CEC module 167. The CEC module 167 may receive and process the collected information and characterize a patient-specific cardiac event based on the collected information. The CEC module 167 may be a processing unit within the controller 151 or microcontroller 162, or may be a separate processor within the NS device 150.

For example, during exercise the CSM module 158 may monitor ST segment changes, and the CEC module 167 may be configured to determine if the monitored ST segment changes are in a patient-specific exercise-related range or exceed a predetermined elevated non-exercise-related threshold. If, for example, physiologic surrogate signals sensed by the PSM module 158 indicate that the patient is currently active, such as exercising, the CEC module 167 may characterize the event as an exercise-induced ischemia. If alternatively the physiologic surrogate signals do not indicate that the patient is exercising, the same level of ST segment changes may be characterized as exceeding a predetermined elevated non-exercise-related threshold, indicating the presence of ischemia and/or MI. The predetermined elevated non-exercise-related threshold may be determined based on a patient-specific historical trend of collected ST segment changes.

Optionally, the NS device may include a neurocardiac therapy (NCT) module 168 configured to deliver a NC therapy based on the cardiac event characterized by the CEC module 167. The NCT module 168 may take various predefined actions based on the particular characterization of the cardiac event. For example, upon the detection of ischemia by the CSM module 158, the NCT module 168 may be configured to deliver NC therapy to treat the ischemia. In another example, upon detection of MI by the CSM module 158 and detection of chest pain by the PSM module 163, the NCT module may be configured to deliver NC therapy configured to provide an anti-ischemic effect without fully masking MI-related chest pain. Even if the CSM module 158 does not detect ischemia or MI, upon the detection of chest pain by the PSM module 163, the NCT module may be configured to deliver a test dose of NC therapy. The test dose of NC therapy may resolve the chest pain, and the effectiveness of the NC therapy at reducing the pain may be used to characterize the chest pain as having a cardiac source (e.g., Cardiac Syndrome X) or having a non-cardiac source (e.g., acid reflux, anxiety, fractured rib, etc.). The NCT module 168 may be a processing unit within the controller 151 or microcontroller 162, or may be a separate processor within the NS device 150. The NCT module 168 may include the stimulation lead 110 and associated circuitry configured to deliver NS through electrodes 111 to the tissue of the patient. Alternatively, or in addition, the NCT module 168 may communicate to a separate IMD or EMD to deliver NS therapy to the patient, such as wirelessly using a receiver/transmitter.

The various modules (e.g., 158, 163, 167, 168) may communicate with each other through the circuitry provided in the NS device 150. Although illustrated as separate components, the modules may share hardware components of the NS device 150. For example, the CSM module 158 may include one or more electrodes 111 for monitoring cardiac signals, and the NCT module 168 also may include the same electrode(s) for delivering NC therapy.

Alternatively, the NS device 150 may receive a communication from an external device and/or another implantable device indicating the detected onset of an ischemia, MI, and/or angina chest pain. For example, the NS device 150 may receive the communication from an implantable pacemaker, ICD, cardiac resynchronization therapy (CRT) device, defibrillator, cardiac rhythm management (CRM) device, and the like. Optionally, the NS device 150 may receive the communication from an external home monitor, external programmer/activator, external ECG monitor, and the like.

A controller/activator device 160 may be implemented to battery 153 of NS device 150 (although a separate recharging device could alternatively be employed). In alternative embodiments, devices separate from the external controller device 160 are employed for charging and programming. A “wand” 165 may be electrically connected to controller device 160 through suitable electrical connectors (not shown). The electrical connectors are electrically connected to coil 166 (the “primary” coil) at the distal end of wand 165 through respective wires (not shown).

The primary coil 166 may be placed against the patient's body immediately above a secondary coil (not shown) integral to the implantable NS device 150. Controller 160 generates an AC-signal to drive current through coil 166 of wand 165. Assuming that primary coil 166 and secondary coil are suitably positioned relative to each other, the secondary coil is disposed within the field generated by the current driven through primary coil 166. Current is then induced in the secondary coil. The current induced in the coil of the NS device 150 is rectified and regulated to battery 153 by recharging circuit 154. Recharging circuit 154 may also communicate status messages to controller 160 during charging operations using pulse-loading and/or any other suitable technique. For example, controller 160 may communicate the coupling status, charging status, charge completion status, etc. Optionally, the controller 160 may operate as a “relay” by receiving cardiac and/or physiologic surrogate signals from a separate IMD and/or EMD and conveying the signals to the NS device 150.

External controller device 160 may also permit the operations of NS device 150 to be controlled by a user (e.g., patient, physician, clinician) after NS device 150 is implanted within the patient. In an exemplary embodiment, the external controller device 160 may be a manual activator used by a patient to designate the presence of chest pain to the NS device 150. Multiple controller devices may be provided for different types of users (e.g., one for the patient and one for a clinician), and optionally the devices may be different depending on the specific user. Controller device 160 can be implemented by utilizing a suitable handheld processor-based system that possesses wireless communication capabilities. Software is typically stored in memory of controller device 160 to control the various operations of controller device 160. Also, the wireless communication functionality of controller device 160 can be integrated within the handheld device package and/or provided as a separate attachable device. The interface functionality of controller device 160 is implemented using suitable software code for interacting with the user and using the wireless communication capabilities to conduct communications with the NS device 150. Controller device 160 preferably provides one or more user interfaces to allow the user to operate the NS device 150 and/or view data stored in the memory 164 of the NS device. The user interface of the controller device 160 may include a display.

FIG. 3 illustrates a diagram of an exemplary system 300 that may be implemented in accordance with an embodiment. The system 300 includes an NS device 302 that is coupled to an NS lead 304 to deliver NC therapies. The system 300 may be implemented using the NS device 150 shown in FIG. 1 in addition to, or instead of, NS device 302. The NS device 302 may include many of the same hardware and/or software components discussed in connection with the NS device 150. The NS lead 304 includes electrodes 306 that are arranged in a two dimensional array of rows 308 and columns 310. The electrodes 306 deliver stimulation pulses based on the current operating NS configuration and parameters. Optionally, other electrode 306 configurations may be used.

The NS device 302 will activate different combinations of the electrodes 306, such as to electronically shift a placement where, and/or configuration at which, an NC therapy is delivered on a particular spinal region. For example, during NS configurations #1, #2 and #3, different electrode column combinations 314, 316 and 318, respectively, may be active. By moving between configurations, and thus active electrode column combinations 314, 316 and 318, the NC therapy can be delivered at different lateral and longitudinal positions along the vertebra relative to a lateral reference point. Similarly, the active electrode row combinations 313, 315 and 317 could be switched to shift a position of the NC therapy in a vertical direction up or down relative to a vertical reference point on a vertebra.

A separate implantable medical device (IMD) 320 may be provided. The IMD 320 may be a pacemaker, anti-tachycardia pacing (ATP) device, ICD device, CRT device, other CRM device such as subcutaneous AF monitor, or other device configured to sense and/or deliver stimulus to cardiac tissue. The IMD 320 is coupled to at least one lead 322 that has a distal end that is configured to be inserted into one or more chambers of the heart. For example, the lead 322 may include a distal end with one or more electrodes 324 inserted into the right ventricle (RV). The lead 322, for example, may also include one or more electrodes 326 located in the right atrium (RA). Optionally, more or different leads may be included, such as leads with electrodes proximate to the left atrium (LA) and/or left ventricle (LV).

The electrodes 324 and 326 sense cardiac signals and may also deliver stimulus and/or pacing to the heart tissue. For example, the electrodes 324 and 326 may sense cardiac electrical signals, cardiac impedance, and/or hemodynamic surrogate signals. The IMD 320 includes a transmitter/receiver (Tx/Rx) 328 that is configured to communicate with a transmitter/receiver (Tx/Rx) 312 in the NS device 302. The Tx/Rx 328 may convey, among other things, cardiac signals sensed at the IMD 320 to the NS device 302. The NS device 302 may analyze the cardiac signals sensed by the electrodes 324, 326 to identify onset, change, and/or termination of a cardiac event, such as MI and/or ischemia. Alternatively, the IMD 320 may perform the analysis of the sensed cardiac signals and communicate orders to the NS device 302 to deliver NC therapy to the patient according to specified parameters. In an alternative embodiment, a single device may be used instead of the separate NS device 302 and IMD 320, where the leads 304 and 322 may extend from a common housing. Various processes are described herein by which the devices of FIGS. 1-3 perform differential analysis and optionally perform therapy.

An external device 340 is shown in FIG. 3. The external device 340 may be a home monitoring device, a Holter monitor worn by the patient, an external IMD or NS programmer, an ECG monitor, and the like. The external device 340 may be similar to the external controller device 160 shown in FIG. 1. The external device 340 may optionally include a display 342, an input keyboard 344, and an antenna 346 used to communicate with the NS device 302 and/or the IMD 320. A surface electrode set 348 is joined to the external device 340 to collect ECG signals as cardiac signals. Optionally, a blood pressure cuff 350 and blood oxygen sensor 352 may be coupled to the external device 340 to sense blood pressure and blood oxygen content, respectively, as physiologic surrogate signals. The external device 340 may transmit cardiac signals and/or physiologic surrogate signals collected from the surface electrode set 348, the blood pressure cuff 350, and/or the blood oxygen sensor 352 to the NS device 302 through the antenna 346.

As described herein, an NS device is used to deliver NS therapy. Optionally, a separate IMD or EMD may be used to deliver NS therapy instead of or in addition to the NS device. The terms “spinal column stimulus”, “SCS” or “NS module” are used herein to collectively refer to any software function, device or system that delivers NS therapy, such as a separate NS device, an IMD configured to deliver NS therapy, an external device configured to deliver NS therapy, and the like. In an embodiment, an NS system for cardiac applications (i.e. ischemia, MI, arrhythmia, angina, etc) is configured to sense at least one of cardiovascular-related physiologic markers or surrogates (e.g., electrogram, systemic hemodynamics, cardiac perfusion, oxygen uptake, and consumption of glyceryl trinitrate, etc.). The system can be incorporated as a transcutaneous device (i.e. patch or other externally wearable device with on-board electronics), a temporary subcutaneous device, or a fully implantable device. The system may be incorporated temporarily or as part of a chronic neurocardiac (NC) therapy or monitoring system for patients with known, or who are at risk for, ischemia.

The NS system may be configured to provide differential diagnosis of events based on whether or not there is the presence of myocardial infarction (MI), ischemia without overt MI, and/or chest pain. The system may use and base the diagnosis on at least one of cardiac electrical signals, hemodynamic surrogates, or other physiologic monitoring via sensors within the system. The system may provide delivery and/or modulation of NC therapies based on the differential diagnosis. In addition to providing therapies, the system may deliver to a treating physician/clinician a detailed diagnostic report of detected episodes/events for use in immediate therapy and/or history trending. For example, the diagnostic reports may compile data collected by sensors within the system and present the data in a convenient readable format for the clinician. In an emergency situation, the diagnostic report may provide the treating physician immediate patient-related information that assists the clinician in making accurate diagnosis and treatment decisions. The diagnostic reports may also be used as evidence that NC therapies are not inadvertently masking ischemic chest pain.

The system may also provide automatic notification of emergency medical personnel/services (EMS) based on the detection of MI, with or without chest pain. Furthermore, the system may include patient notifications. For example, the system may alert a patient experiencing events of MI or non-MI ischemia of the patient's condition, which is especially helpful when the patient does not have accompanying pain, and so is not aware of the ischemia (e.g., silent ischemia). The system may also notify a patient experiencing non-ischemic and non-MI chest pain that the patient should not worry that the pain is ischemia and/or MI-related. Diagnostic output for the patient may be as simple as emitting a red light to indicate an event requiring an emergency response or a green light to indicate that an event is a “stable angina” attack that has been routinely treated. In comparison, the diagnostic output may be more detailed for the clinician, including electrogram activity and/or other physiologic information associated with the onset and evolution of the event.

In an exemplary embodiment, the system may provide differential diagnosis of Cardiac Syndrome X based on detecting a response to cardiac neuromodulation therapy to non-ischemia related pain. In an alternative or additional embodiment, the system may be configured to deliver pain management therapy appropriately when the patient commences exercise. During exercise, ST segment monitoring can be used to determine if the ST segment changes are in a patient-specific “typical range” or at dangerous levels. If the ST segment changes are beyond typical levels, patient alerts may be used to signal the patient to limit exercise.

Differential diagnosis includes measurement and detection. For the differential diagnosis, measurement of a deviation in ST segment may be performed specifically from the same electrodes used to deliver NS therapy. Derived measures from cardiac electrical activity, such as heart rate, heart rate variability, arrhythmia monitoring, QT dispersion, etc., are used primarily for aiding in differential diagnosis, and may also be used to trigger turning NS ON/OFF or for closed-loop titration of NS parameters (e.g., frequency, amplitude, etc.). In addition to cardiac electrical activity, basic physiologic responses from other sensors of the system, such as perspiration, blood pressure, body temperature, cardiac output, activity, etc., are further used as inputs to aid the differential diagnosis of sensed changes. For example, if changes in ST segment are accompanied by hypotension, heart rate elevation, and cold sweats, the event may be diagnosed as treatable.

Therapeutic actions are any events associated with ischemia, with or without MI and regardless of pain. Therapeutic actions may be treated with NC therapy. The parameters of the NC therapy can be tiered based on the severity of the event and by whether physiologic markers of pain are detected.

The use of NC therapy for angina may work at least in part by treating the underlying ischemia that contributes to pain. Successful reduction of ischemia may often be associated with a reduction or elimination of pain, if present. By recruiting collateral circulation, pain may be reduced, and nitrate use may decrease as a result. The treatment of ischemia with NC therapy may reduce some but not necessarily all of true angina/ischemic pain such that true angina pain is not masked by the NC therapy.

Neuromodulation therapy for general non-cardiac pain (i.e. nociception therapy) may operate along different physiological axes to block pain signals from reaching the brain. Nociception or pain therapy treats non-cardiac pain without a direct impact on ischemia or other heart-related events. There is a known risk that nociception therapy may mask true angina pain. Therefore, in an example embodiment, a treatment of chest pain may administer NC therapy prior to nociception therapy. The NC therapy may provide pain relief without masking true ischemia. If the NC therapy does not fully relieve the pain, and the monitored physiologic parameters do not indicate the presence of ischemia, nociception pain therapy may then be applied to relieve the resolving pain.

In an example embodiment, differential diagnosis and tiered NC therapy may be applied during exercise or other physical activities to treat pain and monitor physiologic conditions in an activity-guided therapy. For example, upon commencement of exercise, pain management NS may be automatically “turned on.” Exercise may be detected by one or more indicators, such as activity, minute ventilation, spontaneous heart rate changes, drops in mixed venous saturation, increased cardiac contractility, etc., either alone or in combination. An effect of automatically turning on pain management stimulation may enable the patient to perform daily activities with minimal limitations associated with anginal pain.

During the activity, ST segments may also be monitored to determine if there are ST segment changes. If there are, the system determines whether the ST segment changes are within a “typical range” for the specific patient or are at “extreme” levels. If the ST segment changes are at extreme levels, the patient may be alerted and recommended to limit physical activity to allow the ST segment changes to moderate to the typical range. Furthermore, particularly persistent and/or radical ST segment changes during activity may indicate MI.

FIG. 4 illustrates a flowchart of a detection process 400 for differential analysis of cardiac events. The detection process 400 is implemented by one or more of the NS device 150, NS device 302, IMD 320, external device 340, and the like.

At 402, cardiac signals from the heart are collected to be monitored for indications of at least one of ischemia and MI. The monitoring of cardiac signals may include sensing at least one of cardiac electrical signals, cardiac impedance, and hemodynamic surrogate signals. For example, the monitoring of cardiac signals may include monitoring ST levels in the cardiac signals for ST deviations. For example, ST deviations may be measured and compared to an ST threshold to identify cardiac events and a potential abnormal physiology (e.g., such as when the patient is having an MI, ischemia, a heart block, an arrhythmia, fibrillation, congestive heart failure, and the like).

At 404, physiologic surrogate signals associated with pain are collected to be monitored for indications of chest pain. The monitoring of physiologic surrogate signals may include sensing one or more of blood pressure, heart rate, temperature, perspiration, respiration, chest constriction, etc. These surrogate signals can be measured using electrograms, temperature sensors, and/or impedance, among others.

At 406, the presence or absence of at least one of ischemia and MI is determined based on the monitored cardiac signals collected at 402. Ischemia and/or MI may be detected based at least in part on at least one of severity of ST deviation, duration of ST deviation, and/or changes in at least one of heart rate, heart rate variability, or heart rate morphology. These sensors and surrogates may contribute to the distinction between ischemia that is, or is not, associated with MI. Optionally, other sensors and surrogates may be used in addition to or in place of the provided examples. For example, ischemia or MI may be declared when changes in ST deviation occur that exceed predetermined or programmed threshold levels. For example, the system may record baseline ST levels from one or more cardiac signals. The baseline ST levels may correspond to averages over multiple cardiac cycles. When a new or current ST level exceeds or drops below the baseline ST level by a predetermined amount, the current cardiac event is labeled as ischemic or a MI. The presence of ischemia or MI may be declared i) when a single cardiac event is labeled ischemic or MI, ii) when a series of successive cardiac events are labeled ischemic or MI, or iii) when a predetermined number of cardiac events are labeled ischemic within a predetermined period of time.

At 408, the presence or absence of chest pain is determined based on the monitored physiologic surrogate signals collected at 404. In an example, chest pain may be detected based at least in part on at least one of heart rate, temperature, respiration rate, chest constriction, perspiration, or another physiologic surrogate for pain. For example, the system may record baseline physiologic surrogate levels from one or more physiologic surrogate signals (e.g., baseline heart rate, baseline chest constriction level, etc.). The baseline surrogate levels may correspond to averages over multiple cardiac cycles. When a new or current surrogate level exceeds or drops below the baseline surrogate level by a predetermined amount, the current cardiac event is labeled as chest pain. The presence of chest pain may be declared i) when a single cardiac event is labeled as chest pain, ii) when a series of successive cardiac events are labeled as chest pain, or iii) when a predetermined number of cardiac events are labeled as chest pain within a predetermined period of time. Although the monitored physiologic surrogate signals may be used to detect pain, alternatively or in addition, chest pain may be detected based on a designation by a patient through the use of a manual activator. For example, the patient may have an activator that allows the patient to alert the device or system of pain episodes. Therefore, pain may be diagnosed based on patient input and/or various physiologic surrogate signals.

At 410, a cardiac event (CE) is characterized based on whether the cardiac event occurs in a presence of at least one of ischemia, IM, and chest pain based on the determinations at 406 and 408. For example, the cardiac event characterization may indicate ischemia and chest pain, without MI. In another example, the characterization may indicate MI and ischemia, without pain, which would be a “silent MI.” In an embodiment, specific treatments may be applied depending on the declared characterization of the cardiac event.

The characterization of the CE may be recorded in device memory, such as in a history log, report, patient record, and the like. The CE characterization may include recording the type of CE, the severity, time and/or duration of the CE, and other information of interest. The CE characterization may include storing the associated cardiac signals collected at 402 and/or the surrogate signals collected at 404. The CE characterizations may be saved over a period of time (e.g., days, weeks, months), and then transmitted from the device to a network or an external programmable device. The CE characterizations may be transmitted periodically (e.g., nightly, such as over the Merlin™ network), on demand at the patient's request, on demand at a physician's request, or otherwise.

Once the CE is characterized at 410, the process may end without providing a therapy. Optionally, after characterizing a cardiac event in 410, flow moves from the detection process 400 to a treatment process 500 (shown in FIG. 5) in order to provide specific treatment based on the characterized cardiac event. Although the detection process 400 as illustrated in FIG. 4 includes a specific number of steps, a detection process according to another embodiment may omit steps, rearrange steps, and/or add additional steps.

FIG. 5 illustrates a flowchart of one embodiment of a treatment process 500 for differential analysis of cardiac events. The treatment process 500 may be implemented by one or more of the NS device 150, NS device 302, IMD 320, external device 340, and the like. The treatment process 500 flows to various decision blocks to determine a course of action including treatment and/or notification depending on the particular differential diagnosis of a CE. The treatment process 500 may be implemented based on the CE characterization from the detection process 400 detailed in FIG. 4. For example, the detection process 400 may make a differential diagnosis, then the treatment process 500 may determine which therapeutic and notification actions to take based on the specific analysis.

At 502, a determination is made as to whether the patient is experiencing chest pain. The chest pain was declared based on the physiologic surrogate signals and/or based on patient input using a manual activator, as described in connection with 404 and 408 of the detection process 400. The treatment process 500 provides differential analysis of both pain (“Is this chest pain something to worry about and treat?”) and non-pain event (“Is there silent ischemia occurring?). When the patient is experiencing pain, flow of the process 500 moves along the branch denoted by “Y” to 504.

At 504, the treatment process 500 determines if an actual MI has been detected and declared in the patient. The MI was detected (if present) based on monitored cardiac signals as described in connection with 402 and 406. If an actual MI has been detected, the patient is experiencing chest pain, MI, and ischemia, which is generally present with an MI. In response, flow of the treatment process 500 moves along the branch denoted by “Y” to 506 for treatment of the pain-MI/ischemia.

At 506, the patient is immediately treated for the MI/ischemia, which includes delivering neurocardiac (NC) therapy configured to provide an anti-ischemic effect without fully masking MI-related pain. Alternatively or in addition, pain therapy can be delivered along with a patient notification. Alternatively or in addition, at 506 emergency medical services (EMS) may be immediately notified to respond to the patient. For example, an ambulance may be dispatched to the patient's location. Furthermore, the system may alert the patient that there is an emergency. The alert may tell the patient to respond to the residual chest pain, if he/she has not yet, by taking aspirin, stopping activity, and the like. The alert may also notify the patient that 911 has been called. The treatment at 506 also may include generating an “urgent type” event report and making the event report available for the clinician. The report may be made available to a clinician by transmitting the report to the clinician and/or hospital directly through a network or by transmitting the report to a secure server that is accessible to the clinician prior to the patient arriving at the hospital. For example, access to the report on the server may be password protected, and access to the password limited to the patient, the patient's primary clinician, and/or another designated clinician. In addition, a clinician may also receive an urgent notification, such as through a wireless device carried by the clinician that notifies the clinician that an urgent report has been sent and/or that the patient is experiencing a MI. Therefore, even if the clinician does not receive the detailed urgent event report directly, the clinician may still be notified directly so the clinician can prepare to treat the patient prior to the patient's arrival at the hospital.

The clinician report may include general patient information, such as name, age, sex, height, weight, blood type, current prescribed medication, etc., and history, such as a record of patient illnesses and CEs, record of family history of diseases, record of past prescribed medications, etc. The clinician report may additionally include data obtained from the sensors that monitor cardiac signals and physiologic surrogate signals. Moreover, the report may even feature data from other patients, such as average statistics for the patient's sex and age, that may be obtained externally from databases such as the Merlin™ network. The report may present the collected data as various charts, graphs, histograms, diagrams, pictograms, photographs, and the like to present patient data and comparisons. The report may also display historical trends of data over time on a trend line or other pictorial representation. For example, ECG data obtained from an IMD, such as IMD 320, may be displayed on a report to show ST deviation over time as a trend. The purpose of the urgent clinician report is to provide a single collection of detailed, up-to-date, relevant data to a clinician to allow a treating clinician to make an early, accurate diagnosis and treatment of the patient experiencing MI. After the treatment 506 is administered to the patient, flow of the treatment process 500 proceeds to 522, where the process 500 is complete.

Referring back to 504, if an MI has not been detected or declared, but the patient is experiencing chest pain, flow proceeds along the “N” branch to 508. At 508, a determination is made as to whether ischemia has been detected. Ischemia may be detected, for example, based on the monitored cardiac signals described in connection with 402 and 406 of the detection process 400. If ischemia has been detected, then at this point in the process 400 the patient is experiencing chest pain and ischemia, but not an MI. In response, flow of the treatment process 500 moves along the “Y” branch to 510 for treatment of the pain-ischemia-no MI condition.

At 510, NC therapy is delivered to the patient to treat the ischemia. The NC therapy is configured to lessen or fully resolve the pain. If necessary, additional standard neuromodulation pain (e.g., nociception) therapy may be delivered to further alleviate the cardiac pain. The patient is also issued an alert that “you're OK,” or another message to communicate that the CE characterization is not an emergency situation for now. The alert may also advise the patient that they may wish to rest, but that they need not seek help yet. Additionally, the treatment 510 includes generating a non-urgent, but higher-than-routine-priority, report documenting the event that is then delivered or made available to a clinician. The non-urgent report may optionally include at least most of the same information included in the urgent report discussed above in connection with 506, although the non-urgent report may not be delivered directly to the clinician or include a clinician notification as in the urgent report. Upon administering treatment 510, flow proceeds to 522 where the process 500 terminates.

Referring back to 508, if ischemia has not been detected or declared, flow proceeds along the branch denoted by “N” to 512. At 512, the patient is experiencing actual chest pain but neither MI nor ischemia has been detected. The treatment 512 may include immediately delivering a “pilot” or “test dose” of NC therapy to the patient. A test dose of NC therapy may be appropriate when the detection process 400 detects a pain marker but no markers for ischemia or MI. A test dose is a limited or weaker treatment of NC therapy as compared to a regular dose of NC therapy that is delivered to treat declared MI and/or ischemia. The test does may include a lesser duration of treatment, a lesser intensity of neurostimulation, or both, as compared to the regular dose of NC therapy. If the pain resolves from the test dose of NC therapy, then a suspected diagnosis may be Cardiac Syndrome X. Cardiac Syndrome X (CSX) is likely caused by a disorder of the small arteries in the heart muscle that reduces the amount of blood supplied to the heart muscle. Patients with CSX often experience chest pain during strenuous activities but generally have normal coronary arteries, and there is generally no risk of MI or sudden cardiac death.

In an example embodiment, if the chest pain persists after the test dose of NC therapy is delivered to the patient, then a non-cardiac cause of the chest pain (e.g., acid reflux, anxiety, etc.) is the suspected diagnosis. The treatment 512 may subsequently include delivering nociception therapy, or general non-cardiac neuromodulation pain therapy, to reduce the pain. The patient may also be alerted that “you're OK,” meaning that there is no need to seek help. Furthermore, the CE is recorded in a clinician report that will be available on routine basis to a clinician. Unlike the urgent and non-urgent clinician reports discussed above, after recording this CE, there may be no need to make the report available to the clinician prior to the next scheduled clinical date, such as the next routine doctor's appointment. After treating the patient as described in 512, flow moves along the branch to 522 where the process 500 is complete.

Referring back to 502, if the patient is not experiencing pain, then flow of the treatment process 500 proceeds to 514. At 514, as in connection with 504, a determination is made as to whether an MI has been detected in the patient. If an MI has been detected and declared, flow moves along the branch denoted by “Y” to 506 to treat the MI-no pain condition. In this situation, the patient is treated for the actual MI even though the patient is not experiencing pain and so may not yet be aware of the CE. In general, the treatment 506 for a declared MI may be the same whether or not the patient is also experiencing pain with the MI (i.e. flow along either the “Y” or the “N” branch from decision block 502 may lead to 506 if MI is detected in connection with 504 or 514). As described above, treatment 506 includes delivering NC therapy, notifying emergency medical services, alerting the patient of the emergency, and generating a detailed event report for a treating clinician. After 506, flow proceeds to 522 where the process 500 terminates.

Referring back to 514, if alternatively an MI has not been detected and declared in 514, flow of the treatment process 500 moves along the branch denoted by “N” to 516. At 516, as in connection with 508, a determination is made as to whether ischemia has been detected in the patient. If ischemia has been detected and declared, flow moves along the “Y” branch to treatment 518. In this situation, the patient is not experiencing chest pain or an MI, but is experiencing actual ischemia. Therefore, at 518, NC therapy is delivered to the patient to treat the ischemia-no MI-no pain condition. Since the patient is not experiencing chest pain, the patient may be unaware of the current CE. Furthermore, since the ischemia is not a declared actual MI, the CE is not life-threatening so the patient does not need to be alerted. Still, a non-urgent clinician report of the cardiac event is generated and made available to a treating clinician. Treatment 518 may be similar to treatment in connection with 510, except at 518 the patient need not be alerted because the patient does not experience pain and so may not be aware of the occurrence of the CE. On the other hand, at 510, the patient experiences chest pain and may fear that the pain is a symptom of an MI. Therefore, treatment 510 notifies the patient that “you're OK,” meaning that the pain is not indicative of an actual MI. After treatment 518, flow proceeds down the branch to 522 where the process 500 terminates.

Referring back to 516, if ischemia has not been detected and declared, and the patient is not experiencing chest pain or an MI, flow of the treatment process 500 moves along the “N” branch to 520. At 520, there is no treatment delivered to the patient because there is “nothing wrong.” More specifically, the detection process 400 has not detected the presence of chest pain, MI, or ischemia, so the patient is not experiencing a CE to treat. Therefore, flow proceeds to 522 where the process 500 terminates.

The treatment process 500 may include a tiered automatic notification system that is dependent on the CE characterization as described in 410. The tiered automatic notification system may include patient alerts and/or clinician reports, and may be tiered based on severity of the characterized CE. For example, as shown at 506, 510, and 512, upon detection and declaration of chest pain, the treatment process 500 may issue one of several patient alerts and may generate one of several reports, based on whether ischemia and MI are present concurrently with the chest pain.

The patient alerts may be displayed to the patient on a device, such as external device 340, a personal computer, and/or a wireless phone. The alerts may be tiered such that when a patient experiences chest pain but an MI has not been detected, as in connection with 510 and 512, the patient alert may include the words “You're OK” or related words and/or an indicator such as a green light and/or happy face, to indicate that the CE is not an emergency. Alternatively, the patient alert in connection with 510, where ischemia is detected but not MI, may include the same or similar words but with a different indicator, such as a yellow light and/or an emotionless face. These symbols indicate that the CE is not an emergency, but is more severe than the type of CE treated at 512, where no ischemia is detected. Finally, the most severe CE is at 506, where an actual MI has been detected. The patient alert at 506 is designed to alert the patient of the existence of an emergency situation. The alert may include the word “Emergency,” displayed and/or generated as a voice-recorded sound, and may also notify the patient that emergency medical services have been contacted and are on the way to the patient. The alert optionally or in addition may suggest to the patient to take aspirin, cease activity, and lie down. The alert may include indicator lights (e.g., red light, frowning face, etc.), sounds (e.g., recorded voice, computerized voice, beeps, etc.), and/or vibrations.

The clinician reports, like the patient alerts, that are generated in response to a CE may be tiered in relation to the severity and urgency of the characterized CE. As the CEs increase in severity, the generated clinician reports may be more urgently made available to the clinician. For example, if ischemia has been detected but not MI, as in 510 and 518, a “non-urgent” clinician report may be generated and made available to the clinician. The “non-urgent” report may be made available to a clinician by transmitting the report to the clinician directly, by transmitting the report to a secure server that may be accessed by the clinician, or by storing the report on a transportable memory device that is transported to the clinician. Alternatively, or in addition, both the non-urgent clinician report and the routine clinician report may be stored in the same secure server that is accessible to a treating clinician. Finally, in an urgent situation when a MI has been detected and declared, as in 506, an “urgent” clinician report may be generated and transmitted directly to one or more clinicians and/or the hospital to which the patient will be transported for treatment. Upon transmitting the urgent clinician report, the clinicians/hospital may be notified of receipt of the urgent clinician report and that the patient will be transported to the hospital. In addition, the urgent clinician report may be continuously and/or periodically updated with updated data and information. Therefore, the treating clinicians may have current, detailed information about the patient prior to receiving the patient in person, which could support early, accurate diagnosis and treatment of the MI.

Optionally, the information recorded in the clinician reports may also be tiered to vary based on the urgency and severity of the characterized CE. Although all clinician reports may include at least some of the same information, such as patient information (e.g., name, age, sex, height, weight, blood type, current prescribed medication, etc.), patient history (e.g., record of patient illnesses and CEs, record of family history of diseases, record of past prescribed medications), monitored cardiac signal and physiologic surrogate signal data, and/or information from external databases, the reports may be tiered such that less urgent reports do not include as much detailed information as the more urgent reports. For example, a CE with pain but no detected MI or ischemia, as at 512, may be recorded as an event with a time stamp in a routine clinician report, and may optionally include in the recorded event a chart showing the values of the monitored cardiac and physiologic surrogate signals at that time. At 510 and 518, the non-urgent clinician report may include most, if not all, of the same information as the report generated at 512, including the values of the monitored cardiac and physiologic surrogate signals during the time of the CE. Finally, the urgent clinician report may provide a more detailed recording of the CE, and may also be updated periodically, to provide clinicians treating the patient with MI with current up-to-date information. For example, the urgent report may additionally include both trend lines and single-instant data for most, if not all, of the cardiac and physiologic surrogate signals that are sensed. In addition, the urgent report may collect data related to MI from external sources and databases, such as Merlin™ system, and optionally may compare the collected external data to the data collected internally by the sensors.

In an example embodiment, whenever a patient feels chest pain that is detected and declared, irrespective of the presence of MI or ischemia, the treatment process 500 may provide the patient with a form of neuromodulation therapy and an alert, and may record the cardiac event in a clinician report. Although an example embodiment may provide tiered notifications to a patient, clinician, and/or emergency medical services based on the characterized CE, the notifications are optional. Other examples may not include the same tiered notifications as shown in FIG. 5, or even any notifications.

As shown at 506, 510, 512, and 518, various treatments of the treatment process 500 include delivering NC therapy to the patient. For example, upon detection of ischemia, regardless of whether or not there is MI and also regardless of whether or not there is chest pain, NC therapy is delivered to treat the ischemia, without masking MI-related pain. Successful reduction of ischemia may often be associated with a reduction or elimination of pain, if present.

Although generally NC therapy is used as a cardiac therapy to treat MI and ischemia, NC therapy may also be delivered if only chest pain is detected and declared. For example, at 512, a test dose of NC therapy may be delivered even if MI and ischemia are not detected along with the detection of chest pain. A “test dose” of NC therapy at 512 may imply a smaller dose or amount of NC treatment relative to NC therapies in connection with 506, 510, and 518, where ischemia and/or MI are detected. Although FIG. 5 does not illustrate a differentiation between the doses or amounts of NC treatment between 506, 510, and 518, the parameters of the NC therapies at these treatments, such as duration and intensity, may vary from each other. For example, the parameters of each NC therapy may be adjusted based on severity of the ischemia and/or MI and on the detection of physiologic surrogate signals of pain.

Although the treatment process 500 illustrated in FIG. 5 includes a specific number of steps, a treatment process according to another embodiment may omit steps, rearrange steps, and/or add additional steps.

FIG. 6 illustrates a matrix of a treatment process 600 for differential analysis of cardiac events according to an embodiment. The matrix may be displayed on an external programmer to a user and afford the user the option to change/set the actions taken in connection with each circumstance determined by the differential analysis. The treatment process 500 may be implemented by one or more of the NS device 150, NS device 302, IMD 320, external device 340, and the like. The treatment process 600 may be similar to the treatment process 500 shown in FIG. 5 and/or may be based on the characterization of CEs according to the detection process 400 illustrated in FIG. 4.

As shown in FIG. 6, the matrix of the treatment process 600 includes a horizontal “X” axis 602 that asks “Ischemia?” for whether lschemia has been declared, a vertical “Y” axis 604 that asks “MI?” for whether MI has been declared, and a diagonal “Z” axis 606 that asks “Pain?” for whether chest pain has been declared. The X or “ischemia” axis 602 is bifurcated into a “Yes” column 608 and a “No” column 610 corresponding to the answer of whether ischemia has been declared. Likewise, the Y or “MI” axis 604 is bifurcated into a “Yes” row 612 and a “No” row 614 corresponding to the answer of whether MI has been declared. The intersection of the columns 608, 610 and rows 612, 614 define four quadrants. Quadrant 616 is defined by the intersection of column 608 and row 612. Quadrant 618 is defined by the intersection of column 610 and row 612. The intersection of column 608 with row 614 defines quadrant 620, and quadrant 622 is defined by the intersection of column 610 and row 614.

The Z or “pain” axis 606 extends from the top left to the bottom right of each quadrant, diagonally splitting the four quadrants 616-622 into eight triangle-shaped sub-quadrants 624-638. In each quadrant, the top or right sub-quadrant corresponds to an affirmative answer with regard to whether chest pain has been detected (i.e. yes, chest pain has been detected). Similarly, the bottom or left sub-quadrant in each quadrant corresponds to a negative answer to whether chest pain has been detected (i.e. no, chest pain has not been detected). Whereas the axes 602, 604, and 606 depict CE conditions, the sub-quadrants describe various treatments specific to the particular combination of declared CE conditions.

Quadrant 616 corresponds to detection and declaration of both MI and ischemia. In response, the treatment process 600 immediately delivers NC therapy to treat the MI and associated ischemia and notifies emergency medical services to respond to the patient. In an example, the patient may also be alerted of the emergency condition, and may be advised to cease activity, take aspirin, and lie down. Pain has been declared at sub-quadrant 624 but not at sub-quadrant 626. However, as shown in sub-quadrant 626, the treatment is the same for both 624 and 626, as the treatment for a MI does not depend on the presence or absence of chest pain.

Quadrant 618 corresponds to a detection and declaration of MI but not ischemia. Such a CE consisting of an MI without included ischemia is generally not possible, regardless of whether pain is detected. Therefore, the treatment process 600 does not define specific treatments for sub-quadrants 628 and 630.

If an MI has not been declared but ischemia has, the relevant quadrant is 620, which includes sub-quadrants 632 and 634. In both 632 and 634, because ischemia has been detected, the treatment includes delivery of NC therapy to treat the ischemia, and generation of a non-urgent clinician report of the CE. If chest pain is also detected along with the ischemia, the treatment at sub-quadrant 632 additionally may notify the patient that “you're OK,” or provide another positive indication (e.g., green light, smiley face, etc.) to reassure the patient that the CE is not an MI. On the other hand, if chest pain is not detected, the treatment at sub-quadrant 634 may not necessarily include notifying the patient. There may be no need to notify the patient that “you're OK” at 634 because the patient is not experiencing any chest pain, so the patient is likely not aware of the CE. Therefore, providing a notification to the patient may only cause the patient stress or agitation where there was no stress or agitation prior to the notification. However, alternatively, the patient may prefer to be notified immediately during a CE, even if the patient experiences no pain, so the treatment 634 may include a patient notification similar to the notification at 632.

Sub-quadrants 636 and 638 lie in quadrant 622, which means that neither MI nor ischemia has been declared. If, in addition, no chest pain has been declared, the treatment at sub-quadrant 638 indicates that no treatment need be administered to the patient because “nothing is wrong,” meaning that the patient is not experiencing a CE defined by at least one of an MI, ischemia, and chest pain. At sub-quadrant 628, chest pain is detected although MI and ischemia are not. The treatment 628 includes delivering a test dose of NC therapy in an attempt to diagnose the source or cause of the pain, notifying the patient that “you're OK,” and recording the event in a routine clinician report. If the NC therapy resolves the pain, the suspected diagnosis may be Cardiac Syndrome X, a non-ischemic cardiac condition. If the pain persists after the NC therapy test dose, then a non-cardiac cause of the pain is suspected and the treatment at 628 may subsequently include applying nociception pain therapy to reduce the unresolved pain.

FIG. 7 shows an exemplary IMD 700 that is implanted into the patient as part of an implantable cardiac system. The IMD 700 may be implemented to perform the detection process 400 and/or treatment process 500 shown in FIGS. 4 and 5 respectively. The IMD 700 may be used in combination with, or alternatively in place of, the NS device 150 (shown in FIG. 1), the IMD 320, the NS device 302, and/or the external device 340 (all three shown in FIG. 3). The IMD 700 may be implemented as a full-function biventricular pacemaker, equipped with both atrial and ventricular sensing and pacing circuitry for four chamber sensing and stimulation therapy (including both pacing and shock treatment). Optionally, the IMD 700 may provide full-function cardiac resynchronization therapy. Alternatively, the IMD 700 may be implemented with a reduced set of functions and components. For instance, the IMD may be implemented without ventricular sensing and pacing.

The IMD 700 has a housing 701 to hold the electronic/computing components. The housing 701 (which is often referred to as the “can”, “case”, “encasing”, or “case electrode”) may be programmably selected to act as the return electrode for certain stimulus modes. Housing 701 further includes a connector (not shown) with a plurality of terminals 702, 704, 706, 708, and 710. The terminals may be connected to electrodes that are located in various locations within and about the heart. For example, the terminals may include: a terminal 702 to be coupled to a first electrode (e.g. a tip electrode) located in a first chamber; a terminal 704 to be coupled to a second electrode (e.g., tip electrode) located in a second chamber; a terminal 706 to be coupled to an electrode (e.g. ring) located in the first chamber; a terminal 708 to be coupled to an electrode (e.g. ring) located in the second chamber; and a terminal 710 to be coupled to an electrode (e.g., coil) located in the SVC. The type and location of each electrode may vary. For example, the electrodes may include various combinations of ring, tip, coil and shocking electrodes and the like.

The IMD 700 includes a programmable microcontroller 720 that controls various operations of the IMD 700, including cardiac monitoring and stimulation therapy. Microcontroller 720 includes a microprocessor (or equivalent control circuitry), RAM and/or ROM memory, logic and timing circuitry, state machine circuitry, and I/O circuitry.

IMD 700 further includes a first chamber pulse generator 722 that generates stimulation pulses for delivery by one or more electrodes coupled thereto. The pulse generator 722 is controlled by the microcontroller 720 via control signal 724. The pulse generator 722 is coupled to the select electrode(s) via an electrode configuration switch 726, which includes multiple switches for connecting the desired electrodes to the appropriate I/O circuits, thereby facilitating electrode programmability. The switch 726 is controlled by a control signal 728 from the microcontroller 720.

In the example of FIG. 7, a single pulse generator 722 is illustrated. Optionally, the IMD 700 may include multiple pulse generators, similar to pulse generator 722, where each pulse generator is coupled to one or more electrodes and controlled by the microcontroller 720 to deliver select stimulus pulse(s) to the corresponding one or more electrodes.

Microcontroller 720 is illustrated as including timing control circuitry 732 to control the timing of the stimulation pulses (e.g., pacing rate, atrio-ventricular (AV) delay, atrial interconduction (A-A) delay, or ventricular interconduction (V-V) delay, etc.). The timing control circuitry 732 may also be used for the timing of refractory periods, blanking intervals, noise detection windows, evoked response windows, alert intervals, marker channel timing, and so on. Microcontroller 720 also has an arrhythmia detector 734 for detecting arrhythmia conditions and a morphology detector 736. Although not shown, the microcontroller 720 may further include other dedicated circuitry and/or firmware/software components that assist in monitoring various conditions of the patient's heart and managing pacing therapies.

Microcontroller 720 may also include a cardiac signal monitoring (CSM) module 738 configured to monitor cardiac signals from a patient's heart to detect deviations indicative of at least one of ischemia and MI. The microcontroller 720 additionally may include a pain signal monitoring (PSM) module 740 configured to monitor physiologic surrogate signals associated with pain to detect chest pain. Furthermore, the microcontroller 720 may feature a cardiac event characterization (CEC) module 742 configured to characterize a cardiac event exhibited by the heart based on whether the cardiac event occurs in a presence of at least one of ischemia, MI, and chest pain. The microcontroller 720 may do all of the functions of FIGS. 4 and 5 and combine all of the modules in NS device 150 in FIG. 1.

The CSM module 738, PSM module 740, and/or CEC module 742 may be implemented in hardware as part of the microcontroller 720, or as software/firmware instructions programmed into and executed by the microcontroller 720. Alternatively, the modules 738, 740, and/or 742 may reside separately from the microcontroller 720 as one or more standalone components.

The IMD 700 includes sensing circuitry 744 selectively coupled to one or more electrodes that perform sensing operations through the switch 726 to detect the presence of cardiac activity in the right chambers of the heart. The sensing circuitry 744 may include dedicated sense amplifiers, multiplexed amplifiers, or shared amplifiers. It may further employ one or more low power, precision amplifiers with programmable gain and/or automatic gain control, bandpass filtering, and threshold detection circuit to selectively sense the cardiac signal of interest. The automatic gain control enables the unit 700 to sense low amplitude signal characteristics of atrial fibrillation. Switch 726 determines the sensing polarity of the cardiac signal by selectively closing the appropriate switches. In this way, the clinician may program the sensing polarity independent of the stimulation polarity.

The output of the sensing circuitry 744 is connected to the microcontroller 720 which, in turn, triggers or inhibits the pulse generator 722 in response to the absence or presence of cardiac activity. The sensing circuitry 744 receives a control signal 746 from the microcontroller 720 for purposes of controlling the gain, threshold, polarization charge removal circuitry (not shown), and the timing of any blocking circuitry (not shown) coupled to the inputs of the sensing circuitry.

In the example of FIG. 7, a single sensing circuit 744 is illustrated. Optionally, the IMD 700 may include multiple sensing circuit, similar to sensing circuit 744, where each sensing circuit is coupled to one or more electrodes and controlled by the microcontroller 720 to sense electrical activity detected at the corresponding one or more electrodes. The sensing circuit 744 may operate in a unipolar sensing configuration or in a bipolar sensing configuration.

The IMD 700 further includes an analog-to-digital (ND) data acquisition system (DAS) 750 coupled to one or more electrodes via the switch 726 to sample cardiac signals across any pair of desired electrodes. The data acquisition system 750 is configured to acquire intracardiac electrogram signals, convert the raw analog data into digital data, and store the digital data for later processing and/or telemetric transmission to an external device 754 (e.g., a programmer, local transceiver, or a diagnostic system analyzer). The data acquisition system 750 is controlled by a control signal 756 from the microcontroller 720.

The microcontroller 720 is coupled to a memory 760 by a suitable data/address bus 762. The programmable operating parameters used by the microcontroller 720 are stored in memory 760 and used to customize the operation of the IMD 700 to suit the needs of a particular patient. Such operating parameters define, for example, pacing pulse amplitude, pulse duration, electrode polarity, rate, sensitivity, automatic features, arrhythmia detection criteria, and the amplitude, wave shape and vector of each shocking pulse to be delivered to the patient's heart within each respective tier of therapy.

The operating parameters of the IMD 700 may be non-invasively programmed into the memory 760 through a telemetry circuit 764 in telemetric communication via communication link 766 with the external device 754. The telemetry circuit 764 allows intracardiac electrograms and status information relating to the operation of the IMD 700 (as contained in the microcontroller 720 or memory 760) to be sent to the external device 754 through the established communication link 766.

The IMD 700 can further include magnet detection circuitry (not shown), coupled to the microcontroller 720, to detect when a magnet is placed over the unit. A magnet may be used by a clinician to perform various test functions of the unit 700 and/or to signal the microcontroller 720 that the external programmer 754 is in place to receive or transmit data to the microcontroller 720 through the telemetry circuits 764.

The IMD 700 can further include one or more physiologic surrogate sensors 770. Such sensors are commonly referred to as “rate-responsive” sensors because they are typically used to adjust pacing stimulation rates according to the exercise state of the patient. However, the physiological sensor 770 may further be used to detect changes in cardiac output, changes in the physiological condition of the heart, or diurnal changes in activity (e.g., detecting sleep and wake states). Signals generated by the physiologic sensors 770 are passed to the microcontroller 720 for analysis. The microcontroller 720 responds by adjusting the various pacing parameters (such as rate, AV Delay, V-V Delay, etc.) at which the atrial and ventricular pacing pulses are administered. While shown as being included within the unit 700, the physiologic sensor(s) 770 may be external to the unit 700, yet still be implanted within or carried by the patient. Examples of physiologic surrogate sensors might include sensors that, for example, sense respiration rate, heart rate, pH of blood, blood pressure, temperature, ventricular gradient, activity/exercise, position/posture, minute ventilation (MV), and so forth.

A battery 772 provides operating power to all of the components in the IMD 700. The battery 772 is capable of operating at low current drains for long periods of time, and is capable of providing high-current pulses (for capacitor charging) when the patient requires a shock pulse (e.g., in excess of 2 Å, at voltages above 2 V, for periods of 10 seconds or more). The battery 772 also desirably has a predictable discharge characteristic so that elective replacement time can be detected. As one example, the unit 700 employs lithium/silver vanadium oxide batteries.

The IMD 700 further includes an impedance measuring circuit 774, which can be used for many things, including: lead impedance surveillance during the acute and chronic phases for proper lead positioning or dislodgement; detecting operable electrodes and automatically switching to an operable pair if dislodgement occurs; measuring respiration or minute ventilation; measuring thoracic impedance for determining shock thresholds; detecting when the device has been implanted; measuring stroke volume; and detecting the opening of heart valves; and so forth. The impedance measuring circuit 774 is coupled to the switch 726 so that any desired electrode may be used.

The IMD 700 can be operated as an implantable cardioverter/defibrillator (ICD) device, which detects the occurrence of an arrhythmia and automatically applies an appropriate electrical shock therapy to the heart aimed at terminating the detected arrhythmia. To this end, the microcontroller 720 further controls a shocking circuit 780 by way of a control signal 782. The shocking circuit 780 generates shocking pulses of low (e.g., up to 0.5 joules), moderate (e.g., 0.5-10 joules), or high energy (e.g., 71 to 40 joules), as controlled by the microcontroller 720. Such shocking pulses are applied to the patient's heart through shocking electrodes. It is noted that the shock therapy circuitry is optional and may not be implemented in the IMD 700.

FIG. 8 illustrates a functional block diagram of an external device 800 that is operated in accordance with the processes described herein and to interface with implantable medical devices as described herein. The external device 800 may be a programmer, a workstation, a portable computer, a PDA, a cell phone and the like. Optionally, the external device 800 may be similar to the external device 340. The external device 800 may be configured to perform all functions in FIGS. 4 and 5 and/or coordinate between a NS device and an IMD (e.g., NS device 302 and IMD 320).

The external device 800 includes an internal bus that connects/interfaces with, for example, a central processing unit (CPU) 802, ROM 804, RAM 806, a hard drive 808, a speaker 810, a printer 812, a CD-ROM drive 814, a floppy drive 816, a parallel I/O circuit 818, a serial I/O circuit 820, a display 822, a touch screen 824, a standard keyboard connection 826, custom keys 828, and/or a telemetry subsystem 830. The internal bus is an address/data bus that transfers information between the various components described herein. The hard drive 808 may store operational programs as well as data, such as waveform templates and detection thresholds.

The CPU 802 typically includes a microprocessor, a micro-controller, or equivalent control circuitry, designed specifically to control interfacing with the external device 800 and with an IMD, such as IMD 700. The CPU 802 may include RAM or ROM memory, logic and timing circuitry, state machine circuitry, and I/O circuitry to interface with the IMD 700. The display 822 displays information related to the processes described herein. The display 822 may be connected to a video display 832. The touch screen 824 accepts a user's touch input 834 when selections are made and may display graphic information relating to the IMD 700. The keyboard 826 (e.g., a typewriter keyboard 836) allows the user to enter data to the displayed fields, as well as interface with the telemetry subsystem 830. Furthermore, custom keys 828 turn on/off 838 (e.g., EVVI) the external device 800. The printer 812 prints copies of clinician reports 840 for a physician to review or to be placed in a patient file. Speaker 810 provides an audible alert/warning (e.g., sounds and tones 842) to the user, such as to notify the user that an MI has been detected, as in connection with 506 of the treatment process 500. The parallel I/O circuit 818 interfaces with a parallel port 844. The serial I/O circuit 820 interfaces with a serial port 846. The floppy drive 816 accepts diskettes 848. Optionally, the floppy drive 816 may include a USB port or other interface capable of communicating with a USB device such as a memory stick. The CD-ROM drive 814 accepts CD ROMs 850. Optionally, the CD-ROM drive 814 may be capable of reading/writing DVDs.

The telemetry subsystem 830 includes a central processing unit (CPU) 852 in electrical communication with a telemetry circuit 854, which communicates with both an IEGM circuit 856 and an analog out circuit 858. The circuit 856 may be connected to leads 860. The IEGM circuit 856 is also connected to the implantable leads 860 (e.g., leads 114, 116 and 118 of FIG. 1) to receive and process IEGM cardiac signals as discussed above. Optionally, the IEGM cardiac signals sensed by the leads 860 may be collected by the IMD 700 and then transmitted to the external device 800 wirelessly through an input to the telemetry subsystem 830.

The telemetry circuit 854 is connected to a telemetry wand 862. The analog out circuit 858 includes communication circuits to communicate with analog outputs 864. The external device 800 may wirelessly communicate with the IMD 700 and utilize protocols, such as Bluetooth, GSM, infrared wireless LANs, HIPERLAN, 3G, satellite, as well as circuit and packet data protocols, and the like. Alternatively, a hard-wired connection may be used to connect the external device 800 to the IMD 700.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the invention without departing from its scope. While the dimensions, types of materials and coatings described herein are intended to define the parameters of the invention, they are by no means limiting and are exemplary embodiments. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means—plus-function format and are not intended to be interpreted based on 35 U.S.C. §112, sixth paragraph, unless and until such claim limitations expressly use the phrase “means for” followed by a statement of function void of further structure.

What is claimed is:

1. A method for differential analysis of cardiac events, the method comprising:
monitoring cardiac signals from a heart to detect deviations indicative of at least one of ischemia and myocardial infarction (MI);
monitoring physiologic surrogate signals associated with pain to detect chest pain;
characterizing a cardiac event exhibited by the heart based on whether the cardiac event occurs in a presence of at least one of the ischemia, MI and chest pain.

2. The method of claim 1, wherein the monitoring of cardiac signals includes sensing at least one of cardiac electrical signals, cardiac impedance, and hemodynamic surrogate signals.

3. The method of claim 1, wherein the monitoring of cardiac signals includes monitoring ST levels in the cardiac signals for ST deviations.

4. The method of claim 1, wherein the monitoring of physiologic surrogate signals includes sensing at least one of blood pressure, heart rate, temperature, and respiration.

5. The method of claim 1, further comprising detecting the at least one of ischemia and MI based at least in part on at least one of:
i) severity of ST deviation;
ii) duration of ST deviation; and
iii) changes in at least one of heart rate, heart rate variability, and heart rate morphology.

6. The method of claim 1, further comprising detecting the chest pain based at least in part on at least one of:
i) heart rate;
ii) temperature;
iii) respiration rate;
iv) chest constriction; and
v) perspiration.

7. The method of claim 1, further comprising providing a manual activator configured to permit a patient to designate the presence of the chest pain.

8. The method of claim 1, further comprising, upon detection of ischemia, delivering a neurocardiac (NC) therapy to treat the ischemia.

9. The method of claim 1, further comprising delivering a neurocardiac (NC) therapy.

10. The method of claim 1, further comprising, upon detection of the presence of the chest pain, issuing an alert and generating a report based on whether at least one of ischemia and MI are present concurrent with the chest pain.

11. The method of claim 1, further comprising, when the chest pain is detected and no ischemia is detected, delivering a pain therapy.

12. The method of claim 1, further comprising, when the chest pain is detected and ischemia is detected and no MI is detected, delivering neurocardiac (NC) therapy.

13. The method of claim 1, further comprising, when chest pain, ischemia and MI are detected, delivering a neurocardiac (NC) therapy configured to provide an anti-ischemic effect and notifying patients and physicians that the patient has had an MI.

14. The method of claim 9, wherein the neurocardiac (NC) therapy represents a test dose when a pain marker is detected and no ischemia is detected and no MI is detected.

15. The method of claim 1, further comprising delivering neurocardiac (NC) therapy, wherein parameters of the NC therapy are adjusted based on a severity of the ischemia or MI and based on detection of the physiologic surrogate signals indicative of the pain.

16. A neurocardiac device (NCD) system for differential analysis of cardiac events, the NCD system comprising:
a cardiac signal monitoring (CSM) module configured to monitor cardiac signals from a heart to detect deviations indicative of at least one of ischemia and myocardial infarction (MI);
a pain signal monitoring (PSM) module configured to monitor physiologic surrogate signals associated with pain to detect chest pain; and
a cardiac event characterization (CEC) module configured to characterize a cardiac event exhibited by the heart based on whether the cardiac event occurs in a presence of at least one of the ischemia, MI, and chest pain.

17. The NCD system of claim 16, wherein the CSM module comprises at least one of sensors, electrodes, monitors, and associated software and circuitry.

18. The NCD system of claim 16, wherein the NCD system is configured to be incorporated with a patient as at least one of a transcutaneous device, a temporary subcutaneous device, and a fully implantable device.

19. The NCD system of claim 16, wherein, during exercise, the CSM module is configured to monitor ST segment changes, and the CEC module is configured to determine if the monitored ST segment changes are in a patient-specific exercise-related range or exceed a predetermined elevated non-exercise-related threshold.

20. The NCD system of claim 16, wherein the NCD system further comprises a manual activator configured to permit a patient to designate the presence of chest pain.

21. The NCD system of claim 16, further comprising a neurocardiac therapy (NCT) module configured to deliver a neurocardiac (NC) therapy based on the cardiac event characterized by the CEC module.

22. The NCD system of claim 21, wherein the NCT module is configured to deliver NC therapy upon the detection of ischemia by the CSM module to treat the ischemia.

23. The NCD system of claim 21, wherein, upon detection of MI by the CSM module and detection of chest pain by the PSM module, the NCT module is configured to deliver NC therapy configured to provide an anti-ischemic effect without fully masking MI-related chest pain.

24. The NCD system of claim 21, wherein the NCT module is configured to deliver a test dose of NC therapy upon the detection of chest pain by the PSM module and no detection of ischemia or MI by the CSM module.

25. The NCD system of claim 21, wherein the CSM module comprises an electrode and the NCT module also comprises the electrode, the electrode configured to both monitor cardiac signals and deliver NC therapy.
